(19) United States
(12) Patent Application Publication
(10) Pub. No.: US 2026/0199205 A1
(43) Pub. Date: Jul. 16, 2026

(54) KERATIN FIBRE TREATMENT PROCESS USING A PARTICULAR DIVALENT OR TRIVALENT FATTY ACID SALT AND COMPOSITION COMPRISING SAID SALT

(71) Applicant: L'OREAL, Paris (FR)

(72) Inventor: Christian BLAISE, Aulnay-sous-Bois (FR)

(73) Assignee: L'OREAL, Paris (FR)

(21) Appl. No.: 19/135,644

(22) PCT Filed: Dec. 14, 2023
(86) PCT No.: PCT/EP2023/085876

§ 371 Date: Jun. 4, 2025

(30) Foreign Application Priority Data
Dec. 16, 2022 (FR) FR2213683

(51) Int. Cl. A61K 8/36; A45D 7/04; A61Q 5/12

(52) U.S. Cl. CPC A61K 8/361; A45D 7/04; A61Q 5/12

(57) ABSTRACT

The present invention relates to a process for treating keratin fibres using a particular divalent or trivalent fatty acid salt and to a composition comprising said salt.

### RELATED APPLICATIONS

The present application is a national stage application that claims priority to International Application No. PCT/EP2023/085876 filed Dec. 14, 2023, which claims priority to French Application No. 2213683 filed Dec. 16, 2022, the disclosures of which are incorporated herein by reference in their entirety.

### TECHNICAL FIELD OF THE INVENTION

The present invention relates to a process for treating keratin fibres using a particular divalent or trivalent fatty acid salt and to a composition comprising said salt.

### BACKGROUND OF THE INVENTION

Due to their nature, certain hair types are more sensitive to humidity and may thus have a tendency to form frizziness, which may result in an increase in the volume of the head of hair. These hair types are particularly difficult to style in a humid environment.

In order to remedy this, it is common practice to use hair compositions based on silicone starting materials, which may however leave the hair looking greasy and feeling heavy, or based on oils, the conditioning effect of which may tend to fade rapidly over time. Moreover, consumers are increasingly in search of natural products.

There is thus a real need to develop a hair treatment process that can remedy these drawbacks and that is notably capable of durably limiting the formation of frizziness and/or the increase in volume of the head of hair in a humid environment and of facilitating styling of the hair in a humid environment.

The Applicant has discovered, surprisingly, that all or some of these objectives can be achieved by the process according to the present invention.

### SUMMARY OF THE INVENTION

According to a first aspect, a subject of the present invention is a keratin fibre treatment process comprising step i) or step i′) below:

i) application to the keratin fibres of a composition (A) at acidic pH comprising a) one or more divalent or trivalent salts of fatty acids chosen from the compounds of formula (I) below, isomers thereof, solvates thereof, such as hydrates, and mixtures thereof:

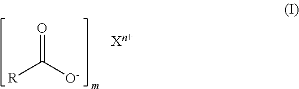

in which formula (I):
   R represents a linear or branched, saturated or unsaturated hydrocarbon-based group comprising from 7 to 29 carbon atoms, the hydrocarbon-based group being optionally substituted with one or more identical or different groups chosen from: —COO⁻ or —OH;
   X represents an alkaline-earth metal, a transition metal, a post-transition metal or a lanthanide;
   m represents a stoichiometric coefficient chosen so as to ensure the electrical neutrality of the compound of formula (I);
   n denotes an integer equal to 2 or 3;
i′) applying to the keratin fibres a composition (B) at acidic pH, comprising:
a′) one or more fatty acids chosen from the compounds of formula (II) below, isomers thereof, solvates thereof such as hydrates, and mixtures thereof:

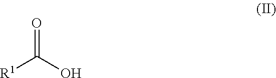

in which formula (II) R¹ represents a linear or branched, saturated or unsaturated hydrocarbon-based group comprising from 7 to 29 carbon atoms, the hydrocarbon-based group being optionally substituted with one or more identical or different groups chosen from: —COOH⁻ or —OH; and
b′) one or more metal salts chosen from mineral or organic salts of alkaline-earth metals, transition metals, post-transition metals, lanthanides, solvates of these salts such as hydrates, and mixtures thereof.

According to a second aspect, a subject of the present invention is a composition (A) or (B) as defined previously.

According to a third aspect, a subject of the present invention is the use of one or more divalent or trivalent salts of fatty acids a) as defined previously or of a composition (A) as defined previously for protecting keratin fibres from moisture, preferably for limiting the formation of frizziness and/or the increase in volume of the head of hair in a humid environment.

According to a fourth aspect, a subject of the present invention is the use of one or more fatty acids a′) as defined previously in combination with one or more metal salts b′) as defined previously or of a composition (B) as defined previously for protecting keratin fibres from moisture, preferably for limiting the formation of frizziness and/or the increase in volume of the head of hair in a humid environment.

### DETAILED DESCRIPTION OF THE INVENTION

For the purposes of the present invention and unless otherwise indicated:
the term “keratin fibres” means fibres of human or animal origin, such as head hair, bodily hairs, the eyelashes, the eyebrows, wool, angora, cashmere or fur. According to the present invention, the keratin fibres are preferably human keratin fibres, more preferentially the head hair.
the term “metal salt” means a salt notably obtained from the action of an acid on a metal.
the term “unsaturated hydrocarbon-based group” means a hydrocarbon-based group comprising one or more conjugated or non-conjugated ethylenic double bonds.
the term “alkyl group” means a saturated, linear or branched hydrocarbon-based radical comprising from 1 to 29 carbon atoms, preferentially from 1 to 25 carbon atoms, more preferentially from 1 to 21 carbon atoms, for example methyl, ethyl, n-propyl, isopropyl, butyl, n-pentyl, n-hexyl, n-decyl, n-undecyl, n-dodecyl, n-tetradecyl, n-hexadecyl or eicosyl.
the term “(Cₓ-C_{y})alkyl group” means an alkyl group comprising from x to y carbon atoms.
the term “isomer” means an optical, geometrical or tautomeric isomer.

The terms “at least one” and “one or more” are synonymous and may be used interchangeably.

### Process for Treating Keratin Fibres

According to a first aspect, a subject of the present invention is a process for treating keratin fibres as defined previously.

The Applicant has found, surprisingly, that the process according to the present invention made it possible to durably limit the formation of frizziness and/or the increase in volume of the head of hair in a humid environment, and to facilitate the styling of the hair in a humid environment.

### First Embodiment

According to a first embodiment, the process comprises step i) of applying to the keratin fibres a composition (A) at acidic pH comprising a) one or more divalent or trivalent salts of fatty acids chosen from the compounds of formula (I) below, isomers thereof, solvates thereof, such as hydrates, and mixtures thereof:

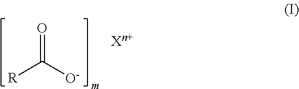

in which formula (I):
   R represents a linear or branched, saturated or unsaturated hydrocarbon-based group comprising from 7 to 29 carbon atoms, the hydrocarbon-based group being optionally substituted with one or more identical or different groups chosen from: —COO⁻ or —OH;
   X represents an alkaline-earth metal, a transition metal, a post-transition metal or a lanthanide;
   m denotes a stoichiometric coefficient chosen so as to ensure the electrical neutrality of the compound of formula (I);
   n denotes an integer equal to 2 or 3.

In formula (I) as defined previously, m=n in the cases where R represents a linear or branched, saturated or unsaturated hydrocarbon-based group comprising from 7 to 29 carbon atoms, not substituted with one or more —COO— groups.

According to a preferred variant of this first embodiment, R represents a linear or branched, saturated or unsaturated hydrocarbon-based group comprising from 7 to 21 carbon atoms, preferably from 9 to 17 carbon atoms, the hydrocarbon-based group being optionally substituted with one or more identical or different groups chosen from: —COO⁻ or —OH.

According to a more preferred variant of this first embodiment, R represents a saturated, linear or branched hydrocarbon-based group comprising from 7 to 29 carbon atoms, preferably from 7 to 21 carbon atoms, more preferentially from 9 to 17 carbon atoms, the hydrocarbon-based group being optionally substituted with one or more identical or different groups chosen from: —COO— or —OH.

According to an even more preferred variant of this first embodiment, R represents a (C7-C29)alkyl group, preferably a (C7-C21)alkyl group, more preferentially a (C9-C17)alkyl group.

According to a preferred variant of this first embodiment, X represents a metal chosen from calcium, magnesium, barium, yttrium, manganese, zinc, bismuth, cerium or samarium, preferably chosen from magnesium, yttrium, zinc, bismuth or cerium, more preferentially chosen from magnesium, yttrium, zinc or bismuth.

The salt(s) a) are preferably chosen from magnesium myristate, zinc myristate, yttrium myristate, cerium myristate, magnesium stearate, zinc stearate, yttrium stearate, bismuth neodecanoate, isomers thereof, solvates thereof such as hydrates and mixtures thereof, preferably chosen from magnesium myristate, zinc stearate, bismuth neodecanoate, yttrium myristate, isomers thereof, solvates thereof such as hydrates and mixtures thereof.

Composition (A) comprises a total salt a) content preferably ranging from 0.5% to 30% by weight, more preferentially ranging from 0.6% to 20% by weight, even more preferentially ranging from 0.8% to 10% by weight, relative to the total weight of composition (A).

### Second Embodiment

According to a second embodiment, the process comprises step i′) of applying to the keratin fibres a composition (B) at acidic pH comprising:
a′) one or more fatty acids chosen from the compounds of formula (II) below, isomers thereof, solvates thereof such as hydrates and mixtures thereof:

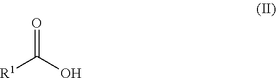

in which formula (II) R¹ represents a linear or branched, saturated or unsaturated hydrocarbon-based group comprising from 7 to 29 carbon atoms, the hydrocarbon-based group being optionally substituted with one or more identical or different groups chosen from: —COOH or —OH; and
b′) one or more salts chosen from mineral or organic salts of alkaline-earth metals, transition metals, post-transition metals, lanthanides, solvates of these salts such as hydrates and mixtures thereof.

Mixing one or more fatty acids a′) with one or more metal salts b′) notably allows the in-situ formation of one or more salts a) as defined previously.

Preferably, the salt(s) b′) are different from the salts a) as defined previously.

According to a preferred variant of this second embodiment, R1 represents a linear or branched, saturated or unsaturated hydrocarbon-based group comprising from 7 to 21 carbon atoms, preferably from 9 to 17 carbon atoms, more preferentially from 11 to 15 carbon atoms, the hydrocarbon-based group being optionally substituted with one or more identical or different groups chosen from: —COOH or —OH.

According to a more preferred variant of this second embodiment, R1 represents a saturated, linear or branched hydrocarbon-based group comprising from 7 to 29 carbon atoms, preferably from 7 to 21 carbon atoms, more preferentially from 9 to 17 carbon atoms, even more preferentially from 11 to 15 carbon atoms, the hydrocarbon-based group being optionally substituted with one or more identical or different groups chosen from: —COOH or —OH.

According to an even more preferred variant of this second embodiment, R1 represents a (C7-C29)alkyl group, preferably a (C7-C21)alkyl group, more preferentially a (C9-C17)alkyl group, even more preferentially a (C11-C15)alkyl group.

The fatty acid(s) a′) are preferably chosen from myristic acid, stearic acid, neodecanoic acid, solvates thereof such as hydrates and mixtures thereof, more preferentially from myristic acid, solvates thereof such as hydrates and mixtures thereof.

According to a preferred variant of this second embodiment, the salt(s) b′) are chosen from the compounds of formula (III) below, isomers thereof, solvates thereof such as hydrates and mixtures thereof:

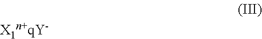

in which formula (III):
X₁ represents an alkaline-earth metal, a transition metal, a post-transition metal or a lanthanide, preferably chosen from calcium, magnesium, barium, yttrium, manganese, zinc, bismuth, cerium or samarium, more preferentially chosen from magnesium, yttrium, zinc, bismuth or cerium, even more preferentially chosen from cerium or yttrium;
n denotes an integer equal to 2 or 3;
Y⁻ represents a monovalent or divalent organic anion preferably comprising 6 or fewer carbon atoms or a mineral anion, preferably a mineral anion;
q denotes a stoichiometric coefficient chosen so as to ensure the electrical neutrality of the compound of formula (III).

According to a preferred variant of this second embodiment, the salt(s) b′) are chosen from the compounds of formula (III) below, isomers thereof, solvates thereof such as hydrates and mixtures thereof:

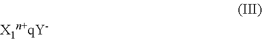

in which formula (III):
   X₁ represents an alkaline-earth metal, a transition metal, a post-transition metal or a lanthanide, preferably chosen from calcium, magnesium, barium, yttrium, manganese, zinc, bismuth, cerium or samarium, more preferentially chosen from magnesium, yttrium, zinc, bismuth or cerium, even more preferentially chosen from cerium or yttrium;
   n denotes an integer equal to 2 or 3;
   Y⁻ represents an anion chosen from: acetate, acetylacetonate, oxalate, gluconate, carbonate, nitrate, sulfate or halides such as chloride, preferably chosen from carbonate, nitrate, sulfate or halides such as chloride, more preferentially chosen from nitrate or halides such as chloride;
   q denotes a stoichiometric coefficient chosen so as to ensure the electrical neutrality of the compound of formula (III).

The salt(s) b′) are preferably chosen from cerium nitrate, cerium chloride, yttrium nitrate, yttrium chloride, solvates thereof such as hydrates and mixtures thereof, more preferentially from cerium nitrate hexahydrate, cerium chloride hexahydrate, yttrium nitrate hexahydrate, yttrium chloride hexahydrate and mixtures thereof.

Composition (B) comprises a total fatty acid a′) content preferably ranging from 1% to 20% by weight, more preferentially ranging from 2% to 10% by weight, relative to the total weight of composition (B).

Composition (B) comprises a total salt b′) content preferably ranging from 0.05% to 0.5% by weight, more preferentially ranging from 0.1% to 0.25% by weight, relative to the total weight of composition (B).

The weight ratio of the total amount of fatty acids a′) to the total amount of salts b′) present in composition (B) is preferably from 5 to 30, more preferentially from 10 to 25.

### Other Characteristics of Compositions (A) or (B)

Composition (A) or (B) is a cosmetic composition, i.e. a composition which comprises a cosmetically acceptable medium, i.e. a medium that is compatible with human keratin fibres.

Composition (A) or (B) may comprise water, preferably in a total content ranging from 5% to 50% by weight, preferably ranging from 7% to 40% by weight, relative to the total weight of composition (A) or (B).

The pH of composition (A) or (B) is acidic. The pH of composition (A) or (B) is preferably from 2 to 5. Preferably, the pH of composition (A) or (B) is equal to 2.

Composition (A) or (B) may comprise one or more organic solvents chosen from linear or branched alkanes containing from 8 to 18 carbon atoms, such as isododecane, tetradecane or isohexadecane, C2-C4 alcohols, such as ethanol, ethers, such as dicaprylyl ether, carbonate esters, such as dicaprylyl carbonate or diethylhexyl carbonate, and mixtures thereof.

When they are present in composition (A), these organic solvents may be present in a content ranging from 20% to 99.9% by weight, relative to the total weight of composition (A).

When they are present in composition (B), these organic solvents may be present in a content ranging from 50% to 95% by weight, preferably ranging from 60% to 95% by weight, relative to the total weight of composition (B).

The pH of composition (A) or (B) may be adjusted using an organic or mineral acid such as hydrochloric acid, sulfuric acid, citric acid, succinic acid, tartaric acid, lactic acid, tosylic acid, benzenesulfonic acid, phosphoric acid or acetic acid. Preferably, the pH of composition (A) or (B) is adjusted with hydrochloric acid.

Composition (A) or (B) may be in any presentation form conventionally used for a haircare application. In a non-limiting manner, composition (A) or (B) may be in the form of a lotion, a cream, a mousse, a gel, a spray or a lacquer.

Preferably, composition (A) or (B) is used as a leave-on application.

Composition (A) or (B) may be packaged in a pump-action bottle or in an aerosol container, so as to apply composition (A) or (B) in vaporized (lacquer) form or in the form of a mousse. In these cases, composition (A) or (B) preferably comprises at least one propellant.

According to a preferred embodiment, composition (A) or (B) comprises a total content of colouring agents of less than 0.1% by weight, preferably less than 0.01% by weight, more preferentially less than 0.001% by weight relative to the total weight of composition (A) or (B); even more preferentially, composition (A) or (B) is free of colouring agents.

The term “colouring agent” means an oxidation dye, a direct dye or a pigment.

The term “oxidation dye” means an oxidation dye precursor chosen from oxidation bases and couplers. Oxidation bases and couplers are colourless or sparingly coloured compounds, which, via a condensation reaction in the presence of an oxidizing agent, give a coloured species.

The term “direct dye” means a natural and/or synthetic dye, including in the form of an extract or extracts, other than oxidation dyes. These are coloured compounds that will spread superficially on the fibre. They may be ionic or nonionic, i.e. anionic, cationic, neutral or nonionic.

According to a preferred embodiment, composition (A) or (B) comprises a total content of silicones of less than 0.1% by weight, preferably less than 0.01% by weight, more preferentially less than 0.001% by weight relative to the total weight of composition (A) or (B); even more preferentially, composition (A) or (B) is free of silicones.

According to a preferred embodiment, composition (A) or (B) comprises a total content of cationic polymers of less than 0.1% by weight, preferably less than 0.01% by weight, more preferentially less than 0.001% by weight relative to the total weight of composition (A) or (B); even more preferentially, composition (A) or (B) is free of cationic polymers.

The term “cationic polymer” means any non-silicone polymer (not comprising silicon atoms) containing cationic groups and/or groups that can be ionized into cationic groups, and not containing any anionic groups and/or groups that can be ionized into anionic groups.

Composition (A) or (B) may be applied to dry or wet keratin fibres, preferably to wet keratin fibres.

The bath ratio of composition (A) or (B) applied to the keratin fibres may range from 0.1 to 10. The term “bath ratio” means the ratio between the total weight of the applied composition (A) or (B) and the total weight of keratin fibres to be treated.

The process may comprise at least one additional step following step i) or i′), chosen from steps ii) and/or iii) below:
ii) a step of applying composition (A) or (B) to the keratin fibres, preferably for a period of at least 10 seconds, said application step optionally being performed under an airtight film of wrapper or plastic film type;
iii) a step of drying the keratin fibres in ambient air or using a heating device.

Preferably, the process comprises the additional steps ii) and iii) as described previously and performed in that order.

The step of applying composition (A) or (B) may last from 10 seconds to 60 minutes, preferably from 30 seconds to 30 minutes.

The temperature of the heating device may range from 45° C. to 230° C., preferably from 45° C. to 100° C., more preferentially from 50° C. to 80° C. A hairdryer, a heating hood, an iron or a heating brush may be used, for example, as heating device.

The drying step iii) may also be a combination of drying with air or with a heating device at a temperature ranging from 50° C. to 80° C., such as a hairdryer or a heating hood, possibly followed by an ironing step, preferably with a straightening iron.

Steps i) or i′) and optionally steps ii) and/or iii) may be repeated as many times as required and each cycle of steps may be spaced from the next by a few minutes to a few days or weeks. Different hair treatments of the invention may be performed between different application cycles, before or after.

### Composition (A) or (B)

According to a second aspect, a subject of the present invention is a composition (A) or (B) as defined previously. The pH of composition (A) or (B) is acidic.

### Uses

According to a third aspect, a subject of the present invention is the use of one or more divalent or trivalent salts of fatty acids a) as defined previously or of a composition (A) as defined previously for protecting keratin fibres from moisture, preferably for limiting the formation of frizziness and/or the increase in volume of the head of hair in a humid environment.

According to a fourth aspect, a subject of the present invention is the use of one or more fatty acids a′) as defined previously in combination with one or more salts b′) as defined previously or of a composition (B) as defined previously for protecting keratin fibres from moisture, preferably for limiting the formation of frizziness and/or the increase in volume of the head of hair in a humid environment.

### Examples

The following examples provide a better understanding of the invention, without, however, being limiting in nature.

The following fatty acid salts a) were used in the examples:

TABLE 1

| Compound | Name |
|---|---|
| Compound a₁ | Magnesium myristate |
| | (CAS: 4086-70-8) |
| Compound a₂ | Zinc stearate |
| | (CAS: 557-05-1) |
| Compound a₃ | Bismuth neodecanoate |
| | (CAS: 34364-26-6) |
| Compound a₄ | Yttrium myristate |

### Synthesis of Compound a₄

Compound a₄ was synthesized according to the procedure described below:

### Procedure

2.5 g (i.e. 0.01 mol) of sodium myristate (M=250.35 g/mol) were placed in a 250 ml flask and 100 ml of absolute ethanol were then added. The mixture was stirred until fully dissolved.

1.2 g (i.e. 0.0031 mol) of yttrium nitrate hexahydrate (M=383.01 g/mol) were then added portionwise and the reaction mixture was then kept stirring at room temperature for 12 hours. The precipitate formed was then filtered off on a sinter funnel and dried under vacuum at 40° C. to constant weight. 2.3 g of yttrium myristate (M=771 g/mol) were thus obtained in the form of a white powder in a yield of 95%. The white solid was characterized by measurement of the infrared spectrum and elemental analysis.

Compositions A1 to A6, C, D1 to D4, B1 to B4 and also the following reference compositions R1 to R4 were prepared and used in the examples:

### Compositions A1 to A6 According to the Invention

TABLE 2

| Ingredients | A1 | | A2 | | A3 | | A4 | | A5 | | A6 | |
|---|---|---|---|---|---|---|---|---|---|---|---|---|
| Compound a₁ | 1 | g | 1 | g | 1 | g | — | | — | | — | |
| Compound a₂ | — | | — | | — | | 1 | g | — | | — | |
| Compound a₃ | — | | — | | — | | — | | 1 | g | — | |
| Compound a₄ | — | | — | | — | | — | | — | | 1 | g |
| Isododecane/ethanol | 100 | ml | | | | | 100 | ml | 100 | ml | 100 | ml |
| mixture (85/15 by | | | | | | | | | | | | |
| mass) | | | | | | | | | | | | |
| Isododecane/ethanol | — | | 100 | ml | | | | | | | | |
| mixture | | | | | | | | | | | | |
| (85/15 by mass) | | | | | | | | | | | | |
| Dicaprylyl ether | | | | | 100 | ml | | | | | | |

| | | | | | | |
|---|---|---|---|---|---|---|
| Aqueous HCl solution | qs pH = | qs pH = | qs pH = | qs pH = | qs pH = | qs pH = |
| | 2 | 2 | 2 | 2 | 2 | 2 |

TABLE 3

| Ingredients | C |
|---|---|
| Myristic acid | 50.3 g (0.2202 mol) |
| Ethanol | 1 L |

TABLE 4

| Ingredients | D1 | D2 | D3 | D4 |
|---|---|---|---|---|
| Cerium nitrate | 1.15 g | — | — | — |
| hexahydrate | (0.00265 mol) | | | |
| Cerium | — | 0.96 g | — | — |
| chloride | | (0.00265 mol) | | |
| hexahydrate | | | | |
| Yttrium nitrate | — | — | 1.01 g | — |
| hexahydrate | | | (0.00265 mol) | |
| Yttrium | — | — | — | 0.80 g |
| chloride | | | | (0.00265 mol) |
| hexahydrate | | | | |
| Water | 100 ml | 100 ml | 100 ml | 100 ml |

### Compositions B1 to B4 According to the Invention

TABLE 5

| Ingredients | B1 | B2 | B3 | B4 |
|---|---|---|---|---|
| Composition C | 80 ml | 80 ml | 80 ml | 80 ml |
| Composition D1 | 20 ml | — | — | — |
| Composition D2 | — | 20 ml | — | — |
| Composition D3 | — | — | 20 ml | — |
| Composition D4 | — | — | — | 20 ml |
| Aqueous HCl | qs PH = 2 | qs pH = 2 | qs pH = 2 | qs pH = 2 |
| solution | | | | |

### Reference Compositions R1 to R4

TABLE 6

| Reference | |
|---|---|
| composition | Ingredients |
| R1 | Ethanol/water mixture |
| | (76/24 by weight) |
| R2 | Isododecane/ethanol mixture |
| | (85/15 by weight) |
| R3 | Isohexadecane/ethanol mixture |
| | (85/15 by weight) |
| R4 | Dicaprylyl ether |

Compositions A1 to A6, B1 to B4 and R1 to R4 were applied and evaluated according to the application and evaluation protocol described hereinbelow.

### Application and Evaluation Protocol

2.7 ml of each of the compositions A1 to A6, B1 to B4 and R1 to R4 were applied to different locks of 2.7 g of Brazilian type IV hair for 30 seconds. The locks were then placed in an oven at 25° C. and 80% humidity at TO and measurements of the volume and frizziness were taken over time up to T0+24H00 by measuring the width of the lock at mid-height.

### Evaluation Results

The results of the measurements of the widths of the locks at mid-height in cm are summarized in the table below:

TABLE 7

| | | Width of locks at | Width of locks at |
|---|---|---|---|
| | Composition | mid-height at TO | mid-height at T0 + |
| Example | tested | (in cm) | 24 h (in cm) |
| 1 | A1 | 2 | 4.5 |
| (Invention) | | | |
| 2 | A2 | 2 | 5 |
| (Invention) | | | |
| 3 | A3 | 2 | 5 |
| (Invention) | | | |
| 4 | A4 | 2 | 5.2 |
| (Invention) | | | |
| 5 | A5 | 2 | 4.5 |
| (Invention) | | | |
| 6 | A6 | 2 | 4.5 |
| (Invention) | | | |
| 7 | B1 | 2 | 4.7 |
| (Invention) | | | |
| 8 | B2 | 2 | 4.5 |
| (Invention) | | | |
| 9 | B3 | 2 | 4.5 |
| (Invention) | | | |
| 10 | B4 | 2 | 5.2 |
| (Invention) | | | |
| 11 | R1 | 2 | 9 |
| (Reference) | | | |
| 12 | R2 | 2 | 9 |
| (Reference) | | | |
| 13 | R3 | 2 | 9 |
| (Reference) | | | |
| 14 | R4 | 2 | 8.5 |
| (Reference) | | | |

It can be seen that the width values of the locks at mid-height are lower for the examples according to the present invention than for the examples using the reference compositions.

The process according to the invention thus makes it possible to limit the formation of frizziness and the increase in volume of the head of hair in a humid environment.

1. A process for treating keratin fibres, comprising step i) or step i′) below:
i) application to the keratin fibres of a composition (A) at acidic pH, composition A comprising a) one or more divalent or trivalent salts of fatty acids chosen from the compounds of formula (I) below, isomers thereof, solvates thereof, hydrates thereof, or mixtures thereof: 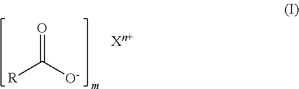
in which formula (I):
R represents a linear or branched, saturated or unsaturated hydrocarbon-based group comprising from 7 to 29 carbon atoms, the hydrocarbon-based group being optionally substituted with one or more identical or different groups chosen from: —COO⁻ or —OH;
X represents an alkaline-earth metal, a transition metal, a post-transition metal or a lanthanide;
m represents a stoichiometric coefficient chosen so as to ensure the electrical neutrality of the compound of formula (I);
n denotes an integer equal to 2 or 3;
i′) applying to the keratin fibres a composition (B) at acidic pH, composition B comprising:
a′) one or more fatty acids chosen from the compounds of formula (II) below, isomers thereof, solvates thereof, hydrates thereof, or mixtures thereof: 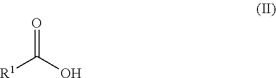
in which formula (II) R¹ represents a linear or branched, saturated or unsaturated hydrocarbon-based group comprising from 7 to 29 carbon atoms, the hydrocarbon-based group being optionally substituted with one or more identical or different groups chosen from: —COOH⁻ or —OH; and
b′) one or more metal salts chosen from mineral or organic salts of alkaline-earth metals, transition metals, post-transition metals, lanthanides, solvates of these salts, hydrates thereof, or mixtures thereof.

2. The process according to claim 1, in which the process comprises step i) and R represents a linear or branched, saturated or unsaturated hydrocarbon-based group comprising from 7 to 21 carbon atoms, the hydrocarbon-based group being optionally substituted with one or more identical or different groups chosen from: —COO⁻ or —OH.

3. The process according to claim 1, in which the process comprises step i) and R represents a saturated, linear or branched hydrocarbon-based group comprising from 7 to 29 carbon atoms, the hydrocarbon-based group being optionally substituted with one or more identical or different groups chosen from: —COO⁻ or —OH.

4. The process according to claim 1, in which the process comprises step i) and X represents a metal chosen from calcium, magnesium, barium, yttrium, manganese, zinc, bismuth, cerium or samarium.

5. The process according to claim 1, in which the process comprises step i) and the salt(s) a) are chosen from magnesium myristate, zinc myristate, yttrium myristate, cerium myristate, magnesium stearate, zinc stearate, yttrium stearate, bismuth neodecanoate, isomers thereof, solvates thereof, hydrates thereof, or mixtures thereof.

6. The process according to claim 1, in which the process comprises step i) and composition (A) comprises a total content of salts a) ranging from 0.5% to 30% by weight, by weight relative to the total weight of composition (A).

7. The process according to claim 1, in which the process comprises step i′) and R¹ represents a linear or branched, saturated or unsaturated hydrocarbon-based group comprising from 7 to 21 carbon atoms, the hydrocarbon-based group being optionally substituted with one or more identical or different groups chosen from: —COOH or —OH.

8. The process according to claim 1, in which the process comprises step i′) and R¹ represents a saturated, linear or branched hydrocarbon-based group comprising from 7 to 29 carbon atoms, the hydrocarbon-based group being optionally substituted with one or more identical or different groups chosen from: —COOH or —OH.

9. The process according to claim 1, in which the process comprises step i′) and the fatty acid(s) a′) are chosen from myristic acid, stearic acid, neodecanoic acid, solvates thereof, hydrates thereof, or mixtures thereof.

10. The process according to claim 1, in which the process comprises step i′) and the salt(s) b′) are chosen from the compounds of formula (III) below, isomers thereof, solvates thereof, hydrates thereof, or mixtures thereof: 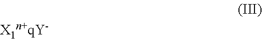
in which formula (III):
X₁ represents an alkaline-earth metal, a transition metal, a post-transition metal or a lanthanide, chosen from calcium, magnesium, barium, yttrium, manganese, zinc, bismuth, cerium or samarium;
n denotes an integer equal to 2 or 3;
Y⁻ represents a monovalent or divalent organic anion comprising 6 or fewer carbon atoms or a mineral anion;
q denotes a stoichiometric coefficient chosen so as to ensure the electrical neutrality of the compound of formula (III).

11. The process according to claim 1, in which the process comprises step i′) and the salt(s) b′) are chosen from the compounds of formula (III) below, isomers thereof, solvates thereof, hydrates thereof, or mixtures thereof: 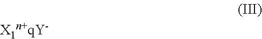
in which formula (III):
X₁ represents an alkaline-earth metal, a transition metal, a post-transition metal or a lanthanide;
n denotes an integer equal to 2 or 3;
Y⁻ represents an anion chosen from: acetate, acetylacetonate, oxalate, gluconate, carbonate, nitrate, sulfate, and halides;
q denotes a stoichiometric coefficient chosen so as to ensure the electrical neutrality of the compound of formula (III).

12. The process according to claim 1, in which the process comprises step i′) and the salt(s) b′) are chosen from cerium nitrate, cerium chloride, yttrium nitrate, yttrium chloride, solvates thereof, hydrates thereof, or mixtures thereof.

13. The process according to claim 1, in which the process comprises step i′) and composition (B) comprises:
a total fatty acid a′) content ranging from 1% to 20% by weight, relative to the total weight of composition (B);
a total salt b′) content ranging from 0.05% to 0.5% by weight, relative to the total weight of composition (B), or a combination thereof.

14. The process according to claim 1, in which the weight ratio of the total amount of fatty acids a′) to the total amount of salts b′) present in composition (B) is from 5 to 30.

15. The process according to claim 1, in which the pH of composition (A) or (B) is from 2 to 5.

16. The process according to claim 1, in which composition (A) or (B) comprises one or more organic solvents chosen from linear or branched alkanes containing from 8 to 18 carbon atoms, C₂-C₄ alcohols, or mixtures thereof.

17. The process according to claim 1, in which composition (A) or (B) is applied to dry or wet keratin fibres.

18. The process according to claim 17, comprising at least one additional step successive to step i) or i′), chosen from the following steps ii), iii), or a combination thereof:
ii) a step of applying composition (A) or (B) to the keratin fibres, preferably for a period of at least 10 seconds, said application step optionally being performed under airtight conditions;
iii) a step of drying the keratin fibres in ambient air or using a heating device.

19. A composition for treating keratin fibres selected from the group consisting of compositions (A), (B) or a combination of (A) and (B), each of (A) and (B) as defined in claim 1.

20. A method for protecting keratin fibres from moisture, comprising use on the keratin fibres of one or more divalent or trivalent salts of fatty acids a) or of a composition (A), each of a), and (A) as defined in claim 1, for protecting limiting the formation of frizziness and increase in volume of the head of hair in a humid environment.

21. The method according to claim 20, comprising use on the keratin fibres of one or more fatty acids a′) in combination with one or more metal salts b′) or of a composition (B), each of a′), b′) and (B) as defined in claim 1, for limiting the formation of frizziness and increase in volume of the head of hair in a humid environment.
